(19) United States
(12) Patent Application Publication
(10) Pub. No.: US 2024/0423630 A1
(43) Pub. Date: Dec. 26, 2024

(54) OCCLUSION APPARATUS AND METHODS

(71) Applicant: Medtronic, Inc., Minneapolis, MN (US)

(72) Inventor: Grant Alexander MENON, Santa Rosa, CA (US)

(73) Assignee: Medtronic, Inc., Minneapolis, MN (US)

(21) Appl. No.: 18/614,990

(22) Filed: Mar. 25, 2024

Related U.S. Application Data
(60) Provisional application No. 63/523,017, filed on Jun. 23, 2023.

(51) Int. Cl. A61B 17/12; A61B 17/00

(52) U.S. Cl. CPC A61B 17/12122; A61B 17/00234; A61B 17/0057; A61B 2017/00243; A61B 2017/00632; A61B 2017/12059

(57) ABSTRACT

Occlusion apparatus comprise a radially expandable frame extending along a frame axis from a first axial end to a second axial end. The occlusion apparatus further comprise a first lattice of sutures extending across a first opening defined by the first axial end of the radially expandable frame. Methods are also provided that radially expand a frame from a radially collapsed orientation to a radially expanded orientation after retracting a capsule. Methods further comprise filling an interior area of the radially expandable frame with support material to facilitate maintenance of the radially expandable frame in a radially expanded orientation. The first lattice of sutures inhibits the support material from exiting the interior area through the first opening.

### CROSS-REFERENCE TO RELATED APPLICATIONS

This application claims the benefit of U.S. Provisional Patent Application Ser. No. 63/523,017, filed Jun. 23, 2023, the entire content of which is incorporated herein by reference.

### FIELD

The present disclosure relates generally to occlusion apparatus and methods and, more particularly, to occlusion apparatus including a radially expandable frame and methods of deploying an occlusion apparatus including radially expanding the radially expandable frame from a radially collapsed orientation to a radially expanded orientation.

### BACKGROUND

A human heart includes the left atrial appendage which is a small organ attached to the left atrium of the heart. Normally, the left atrial appendage contracts and forces blood into the left atrium. However, in persons suffering from atrial fibrillation, the contractions of the left atrial appendage are not regular which can lead blood to pool within the left atrial appendage. Furthermore, the pooling of blood in the left atrial appendage is conducive to thrombi formation. Once thrombi are formed, they may leave the left atrial appendage and form an embolus within a patient's blood stream. Eventually, the embolus may obstruct a vessel leading to a heart attack or a stroke.

It is known to provide anticoagulants (i.e., blood thinners) to atrial fibrillation patients to reduce their risk of stroke. But while these drugs are known to reduce clot formation, they are also known to increase bleeding complications and can cause adverse reactions for some patients. Further, anticoagulants can cause adverse reactions with other medications a patient may take.

Recently, various occlusion devices have been developed which can be positioned within the left atrial appendage which serve to close the left atrial appendage off to the left atrium. Once in place, new tissue may grow over the exposed surface(s) of the occlusion device which has the effect of removing the left atrial appendage from the circulatory system and thus reducing or eliminating emboli therefrom.

Depending upon the patient, the size of one's left atrial appendage can vary significantly. Consequently, previously developed left atrial appendage occlusion devices may create problems such as poor fit to the left atrial appendage. And even if a left atrial appendage occlusion device does fit within a patient's left atrial appendage, the device can still experience poor sealing at its edges which can lead to leakage. There is a need to provide improved medical devices and methods that overcome the drawbacks of previous devices and reliably reduce patients' risk of a stroke or a heart attack.

### SUMMARY

The following presents a simplified summary of the disclosure to provide a basic understanding of some aspects described in the detailed description.

In aspects, occlusion apparatus comprise a radially expandable frame extending along a frame axis from a first axial end to a second axial end. The occlusion apparatus further comprise a first lattice of sutures extending across a first opening defined by the first axial end of the radially expandable frame.

In further aspects, methods are provided of deploying an occlusion apparatus comprising a radially expandable frame extending along a frame axis from a first axial end to a second axial end. The occlusion apparatus further comprises a first lattice of sutures extending across a first opening defined by the first axial end of the radially expandable frame. The radially expandable frame is positioned in a radially collapsed orientation within an interior area of a capsule of a delivery device. The methods comprise retracting the capsule in a proximal direction relative to the radially expandable frame such that the radially expandable frame is positioned outside of the capsule. The methods further comprise radially expanding the radially expandable frame from the radially collapsed orientation to a radially expanded orientation. The methods further comprise filling an interior area of the radially expandable frame with a support material to facilitate maintenance of the radially expandable frame in the radially expanded orientation. The first lattice of sutures inhibits the support material from exiting the interior area through the first opening.

Additional features and advantages of the aspects disclosed herein will be set forth in the detailed description that follows, and in part will be clear to those skilled in the art from that description or recognized by practicing the aspects described herein, including the detailed description which follows, the claims, as well as the appended drawings. It is to be understood that both the foregoing general description and the following detailed description present aspects intended to provide an overview or framework for understanding the nature and character of the aspects disclosed herein. The accompanying drawings are included to provide further understanding and are incorporated into and constitute a part of this specification. The drawings illustrate various aspects of the disclosure, and together with the description explain the principles and operations thereof.

### BRIEF DESCRIPTION OF THE DRAWINGS

These and other features, aspects and advantages are better understood when the following detailed description is read with reference to the accompanying drawings, in which:

FIG. 1 is a schematic view of an occlusion apparatus according to aspects of the disclosure being deployed within a left atrial appendage of a heart;

FIG. 2 is a perspective view of a radially expandable frame in accordance with aspects of the disclosure;

FIG. 3 is a perspective view of a radially expandable frame in accordance with further aspects of the disclosure;

FIG. 4 is a cross-sectional view of the radially expandable frame along line 2-2 of FIG. 2 wherein an interior area of the radially expandable frame is filled with a support material;

FIG. 5 illustrates an end view of the radially expandable frame along lines 5A-5A and/or 5B-5B of FIG. 4 in accordance with aspects of the disclosure;

FIG. 6 illustrates an end view of the radially expandable frame along lines 5A-5A and/or 5B-5B of FIG. 4 in accordance with further aspects of the disclosure;

FIG. 7 illustrates an end view of the radially expandable frame along lines 5A-5A and/or 5B-5B of FIG. 4 in accordance with still further aspects of the disclosure;

FIG. 8 illustrates a ring of a first axial end and/or a second axial end in a collapsed orientation in accordance with aspects of the disclosure;

FIG. 9 illustrates a ring of a first axial end and/or a second axial end in an expanded orientation in accordance with further aspects of the disclosure;

FIG. 10 illustrates the ring of FIG. 9 in a collapsed orientation;

FIG. 11 is a cross-sectional schematic view of a handle apparatus of a delivery device in accordance with aspects of the disclosure;

FIG. 12 schematically illustrates a radially expandable frame in a radially collapsed orientation within an interior area of a capsule of a delivery device;

FIG. 13 schematically illustrates enlarged portions of the delivery device taken at view 13 of FIG. 12; and

FIGS. 14-19 illustrate example steps in methods of deploying an occlusion apparatus in accordance with aspects of the disclosure.

perspective view of a radially expandable frame in accordance with aspects of the disclosure.

### DETAILED DESCRIPTION

Aspects will now be described more fully hereinafter with reference to the accompanying drawings in which example aspects are shown. Whenever possible, the same reference numerals are used throughout the drawings to refer to the same or like parts. However, this disclosure may be embodied in many different forms and should not be construed as limited to the aspects set forth herein.

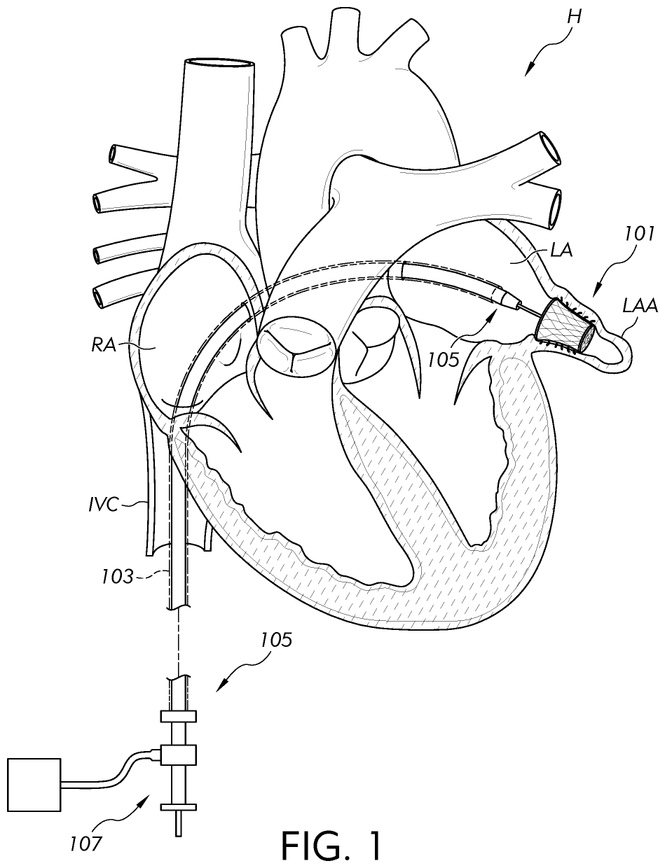

FIG. 1 illustrates an example human heart “H” including a left atrium “LA” with a left atrial appendage “LAA”. An occlusion apparatus 101 of aspects of the disclosure is schematically illustrated implanted within the left atrial appendage “LAA” at the entrance of the left atrial appendage “LAA”. To place the occlusion apparatus 101, an introducer sheath 103 can be used to guide a delivery device 105 carrying the occlusion apparatus 101 to the target site of the left atrial appendage “LAA”. For example, the introducer sheath 103 can be introduced into the right atrium “RA” from the inferior vena cava “IVC”. The introducer sheath 103 can then pass through the wall of the right atrium “RA” the wall of the left atrium “LA” to place the distal opening of the introducer sheath 103 within the left atrium “LA”. An access lumen is thereby created by the introducer sheath 103 to deliver the occlusion apparatus 101 with the delivery device 105 to the target site of the left atrial appendage “LAA”. As schematically illustrated in FIG. 1, the delivery device 105 can comprise a handle apparatus 107 that can be manipulated by a surgeon outside of the patient's vasculature to control delivery and deployment of the occlusion apparatus 101.

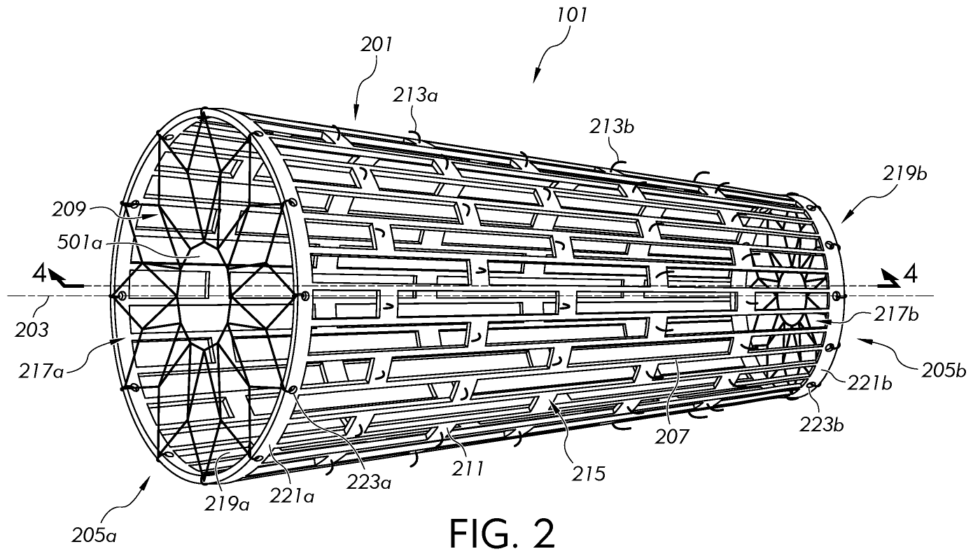

An example of the occlusion apparatus 101 is shown in FIG. 2. The occlusion apparatus 101 can comprise a radially expandable frame 201 extending along a frame axis 203 from a first axial end 205a to a second axial end 205b. The frame can comprise a wide variety of structural arrangements and be formed from a wide range of materials to allow the frame to expand (e.g., self expand) from a radially collapsed orientation to a radially expanded orientation. For example, the illustrated radially expandable frame 201 comprises a self expanding frame comprising a shape memory material that allows the frame to self expand when being deployed. In some embodiments, the radially expandable frame 201 can comprise nitinol or other shape memory material.

As illustrated, in some embodiments, the radially expandable frame can comprise a plurality of longitudinal segments 207 extending in a direction of the frame axis 203. In some embodiments, as shown, the longitudinal segments 207 can be extended as an array of longitudinal segments 207 that surround the frame axis 203 to at least partially define an interior area 209. Further, as shown, a plurality of axially spaced arrays of tabs 211 surrounding the frame axis 203 can attach adjacent longitudinal segments 207 together. To enhance compressibility, the tabs of one array can be axially offset from the tabs of an adjacent array to enhance compressibility. With the illustrated construction, the radially expandable frame 201 can comprise a plurality of rectangular openings although other shaped openings may be provided in further embodiments. For example, the radially expandable frame may be provided by a mesh construction, or non-linear segments such as curved segments. In some embodiments, the segments can comprise curved segments that undulate along the length of the segment (e.g., in a sinusoidal shape).

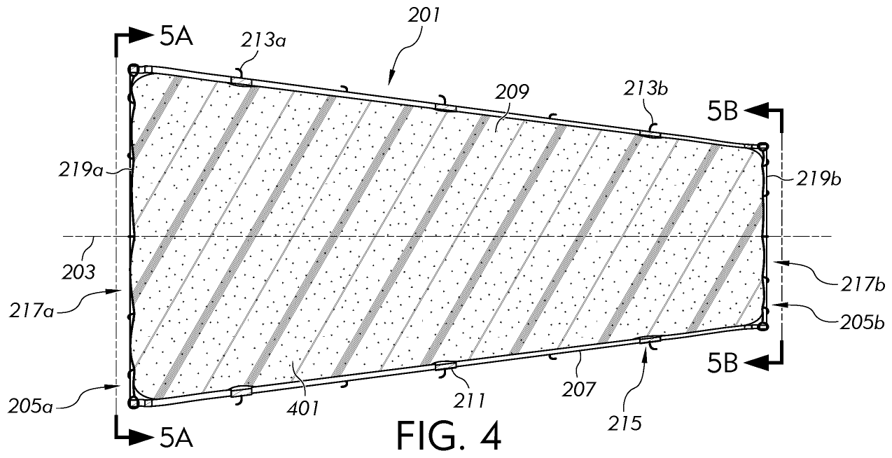

As illustrated, the radially expandable frame 201 can comprise a frustoconical shape such as the illustrated circular frustoconical shape. As will be appreciated by the cross-section of FIG. 4, in some embodiments, the frustoconical shape of the radially expandable frame 201 can comprise a continuous taper where the outer diameter is directly proportional to a distance from one of the axial ends of the radially expandable frame 201. In the illustrated embodiment, the frustoconical shape can taper in a direction along the frame axis 203 from the first axial end 205a toward the second axial end 205b. Providing the frustoconical shape that tapers in the direction along the frame axis 203 from the first axial end 205a toward the second axial end 205b can be beneficial, for example with an application wherein a void to be filled tapers in the same direction. For example, as shown in FIG. 1, in some heart anatomies, the left atrial appendage “LAA” can taper in a direction away from the left atrium “LA”. As such, tapering the frustoconically shaped radially expandable frame 201 from the first axial end 205a toward the second axial end 205b allows the radially expandable frame 201 to become wedged within the left atrial appendage “LAA” with the larger diameter first axial end 205a disposed near the entrance to the left atrial appendage “LAA”.

Although not shown, the frustoconical shape may taper in the opposite direction in further embodiments to accommodate alternative anatomical features. Still further, in some embodiments, the frustoconical frame may comprise a circular cylindrical or other curvilinear cylindrical shape that may or may not be frustoconical.

In some embodiments, the occlusion apparatus 101 can comprise further anchoring features designed to facilitate maintenance of the occlusion apparatus 101 in the desired position. For example, the radially expandable frame can comprise a plurality of protrusions such as the illustrated hooks although other protrusions can comprise pins, nubs, barbs, or teeth designed to help prevent an undesired relative movement between the occlusion apparatus and the anatomy that the occlusion apparatus engages. As shown in FIG. 2, the radially expandable frame 201 can comprise a plurality of protrusions 213a, 213b extending away from the frame axis 203 and an exterior 215 of the radially expandable frame 201. As shown in FIG. 2, in some embodiments, each protrusion 213a of a first set of the plurality of protrusions can comprise a distal end extending in a direction towards the first axial end 205a of the radially expandable frame 201. Referring to FIG. 1, each protrusion 213a of the first set of the plurality of protrusions with the distal end extending in the direction towards the first axial end 205a would help maintain the installed position of the radially expandable frame 201 by resisting movement of the radially expandable frame 201 out of the left atrial appendage “LAA” and into the left atrium “LA”. Furthermore, each protrusion 213b of the second set of the plurality of protrusions with the distal end extending in a direction towards the second axial end 205b can help maintain the installed position of the radially expandable frame 201 by resisting movement of the radially expandable frame 201 further into the left atrial appendage “LAA” where an additional undesired cavity may form between the left atrium “LA” and the first axial end 205a that may result in thrombi formation that the occlusion apparatus was designed to prevent.

As further illustrated in FIGS. 2 and 4, a first lattice 217a of sutures extend across a first opening 219a defined by the first axial end 205a of the radially expandable frame 201. Although not required, in some embodiments, the second axial end 205b may define a second opening 219b. If provided with a second opening, in some embodiments, a second lattice 217b of sutures can also extend across the second opening 219b although other closures can be provided in further embodiments. The first lattice 217a of sutures, and if provided, the second lattice 217b of sutures, are designed to extend across the respective openings to inhibit support material from exiting the interior area 209 through the corresponding openings 219a, 219b of the radially expandable frame 201 in the radially expanded orientation. Each lattice of sutures can be attached at circumferential locations of the radially expandable frame 201 about the first axial end 205a and/or the second axial end 205b. For example, with reference to FIG. 2, the first axial end 205a can comprise a first ring 221a defining the first opening 219a. The first lattice 217a of sutures can be attached to the first ring 221a at a plurality of first ring locations 223a that are circumferentially arranged about the frame axis 203. For example, as shown, the first ring locations can comprise a plurality of circumferentially arranged apertures. The second axial end 205b can comprise a second ring 221b defining the second opening 219b. The second lattice 217b of sutures can attached to the second ring 221b at a plurality of second ring locations 223b that are circumferentially arranged about the frame axis 203. For example, as shown, the second ring locations can comprise a plurality of circumferentially arranged apertures. Throughout the disclosure, sutures refer to surgical sutures but can also include other sutures such as filaments, wires, strings, or strands of polymer, metal, or any suitable material.

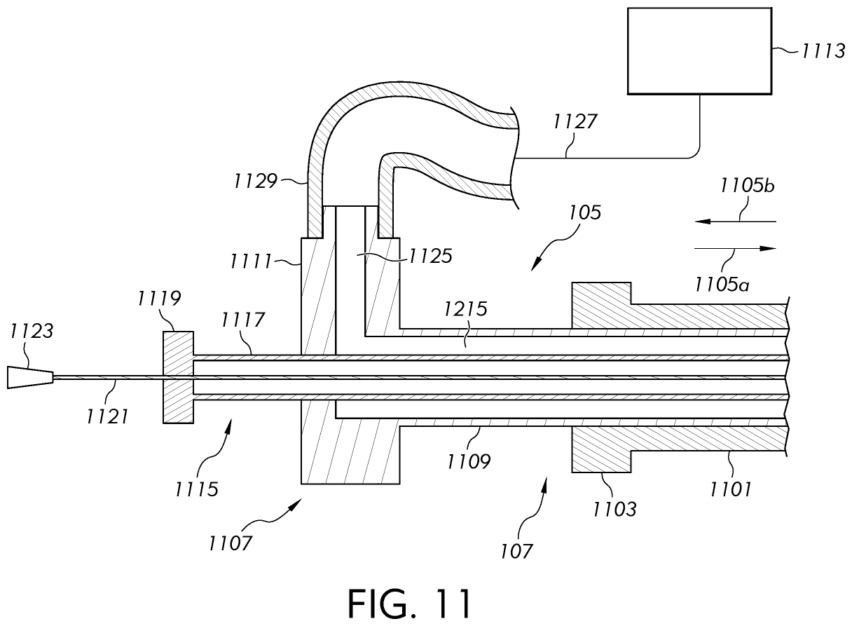

As shown in the figures, the rings, if provided, can have a thickness that is relatively thin in a radial direction extending perpendicular to the frame axis 203 compared to the length of the ring extending substantially parallel to the frame axis 203. Orienting the ring such that it is relatively thin in the direction perpendicular to the frame axis 203 can reduce the bending moment of inertia; thereby reducing the difficulty of collapsing the frame to load the capsule of the delivery device with the occlusion apparatus in the collapsed orientation. FIG. 8 illustrates a schematic end view of the first ring 221a in the collapsed orientation where the first ring is shown buckled in the radial direction due to the relatively thin thickness being directed in the radial direction perpendicular to the frame axis.

In some embodiments, one ring can include a ring stiffness that is greater than a ring stiffness of the other ring. For instance, in the illustrated embodiment, the ring stiffness of the second ring 221b can be greater than the ring stiffness of the first ring 221a to facilitate greater collapsing of the first ring that has a larger diameter due to the frustoconical shape of the radially expandable frame 201. For purposes of this application, ring stiffness is determined by placing a ring between two pieces of stainless steel. Then applying a weight with a resultant vector extending through the central axis of the ring until the distance between the two plates is reduced by 1 millimeter. The same test is performed for the other ring with the ring having the highest weight to achieve the 1 millimeter reduction in plate distance having the highest ring stiffness.

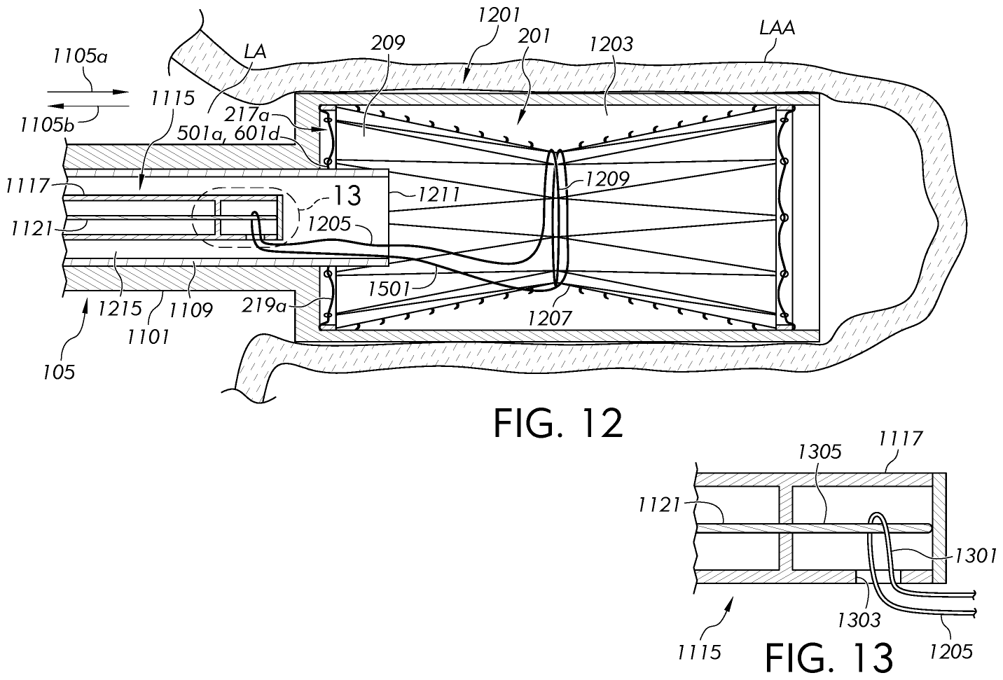

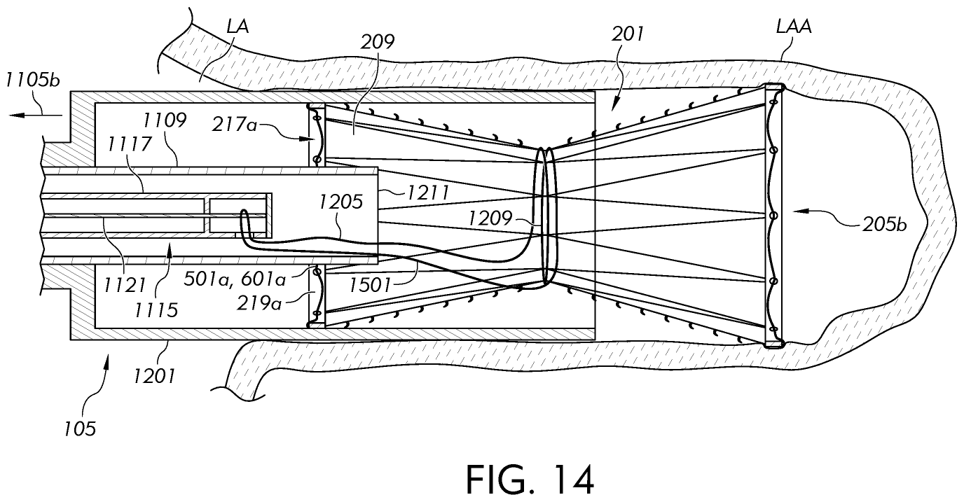

FIGS. 9 and 10 illustrate an alternative embodiment of a first ring 921a that may be used to achieve greater collapsing of the first ring that has a larger diameter than the second ring. As shown, the first ring 921a can comprise a plurality of linked segments 923a-d that may be linked together at corresponding ends by fasteners 925 extending through slots (not shown). The first ring 921a may dilate to the radially expanded orientation shown in FIG. 9 or contract to the radially collapsed orientation shown in FIG. 10.

Turning back to FIG. 2, the first and second rings 221a, 221b comprise corresponding first and second ring locations 223a, 223b (e.g., apertures) that provide attachment locations for the first and second lattices 217a, 217b of sutures. The apertures can be directed to extend substantially perpendicular to the frame axis 203 due to the orientation of the thickness of the rings wherein the sutures thread through the apertures and around the outer edge to extend across the corresponding opening 219a, 219b, over the outer edge again and back through the next aperture and back and forth until the lattice is formed.

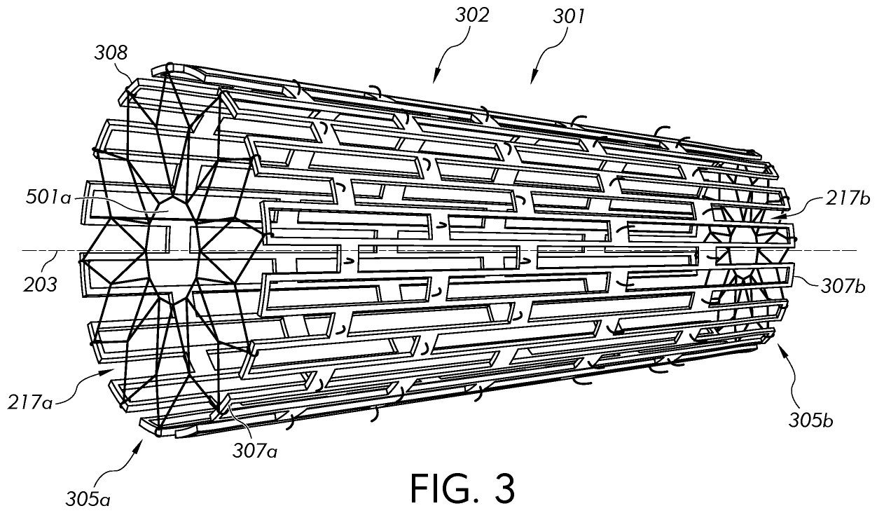

FIG. 3 illustrates an alternative embodiment of an occlusion apparatus 301 that, unless otherwise noted, can be identical to the occlusion apparatus 101 discussed throughout the disclosure. As shown, the first axial end 305a and/or the second axial end 305b of a radially expandable frame 302 can comprise a plurality of crowns 307a, 307b that are each circumferentially arranged about the frame axis 203. The plurality of crowns 307a, 307b provide attachment locations for the first and second lattices 217a, 217b of sutures. For example, the first axial end 305a can comprise a first plurality of crowns 307a circumferentially arranged about the frame axis 203, wherein the first lattice 217a of sutures are threaded through the first plurality of crowns 307a. Furthermore, in the illustrated example, with the second lattice 217b of sutures, the second axial end 305b can comprise a second plurality of crowns 307b circumferentially arranged about the frame axis 203, wherein the second lattice 217b of sutures are threaded through the second plurality of crowns 307b. The illustrated crowns show an outer rim 308 forming the distal end of the crown and circumferentially spaced about the openings of the radially expandable frame 302 of the occlusion apparatus 301. The sutures extend through the crown by extending through the distal end of the opening adjacent the outer rim 308 with the suture wrapping around the outer rim to pass over the opening of the axial end to another crown of the axial end. In some embodiments, the crowns can be formed as separate distinct extensions with distinct eyelets. Providing the distal ends with a plurality of crowns, rather than rings, can help reduce the rigidity of the occlusion apparatus at the axial ends of the radially expandable frame and therefore simplify radial collapsing of the radially expandable frame into the capsule of the delivery device. Throughout the disclosure, reference of various features of the occlusion apparatus and the method of deploying the occlusion apparatus will be described with reference to the radially expandable frame 201 of FIG. 2 with the understanding that such description equally applies to features and methods associated with the radially expandable frame 302 of FIG. 3 unless otherwise noted.

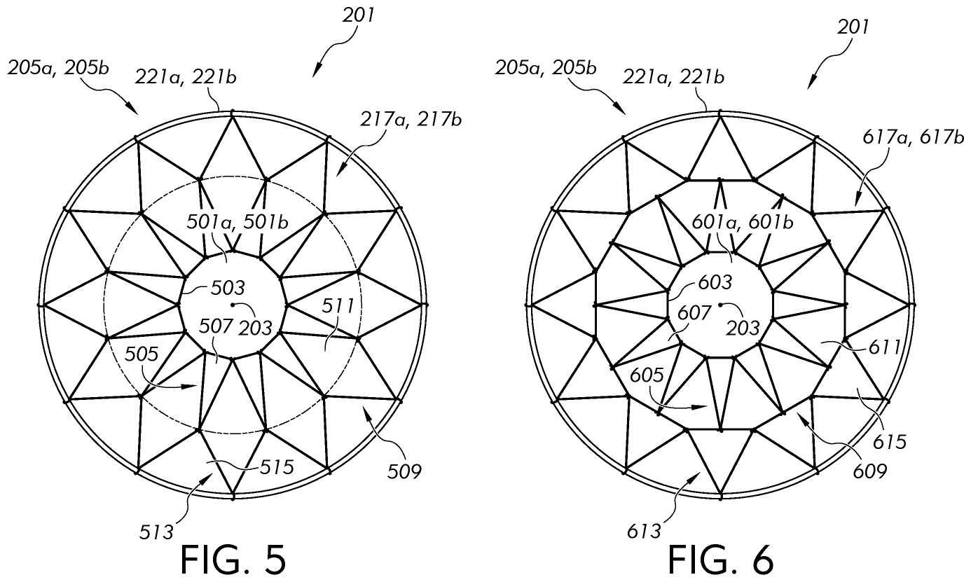

FIG. 5, illustrates features of the first lattice 217a and the second lattice 217b of sutures of the first and second axial ends 205a, 205b of the radially expandable frame 201 in accordance with one embodiment of the disclosure. The diameter of the rings 221a, 221b may differ depending on whether there is a difference in diameter between the rings of the first axial end and the second axial end. For example, as shown in FIG. 2, it is understood that the second axial end 205b comprises a second ring 221b with a smaller diameter than the first ring 221a of the first axial end 205a. However, to simplify discussion, the features of the lattices will be discussed with the understanding that these features may apply to a wide range of ring diameters. As shown, the first lattice 217a of sutures can define a central first lattice opening 501a and the second lattice 217b of sutures can define a central second lattice opening 501b. Each central lattice opening 501a, 501b can be substantially concentric with the frame axis 203. A plurality of substantially straight central segments in tension can be aligned in a circular array to approximate a circular central lattice opening with the substantially straight segments. Although not shown, other central lattice opening shapes may be provided in further embodiments.

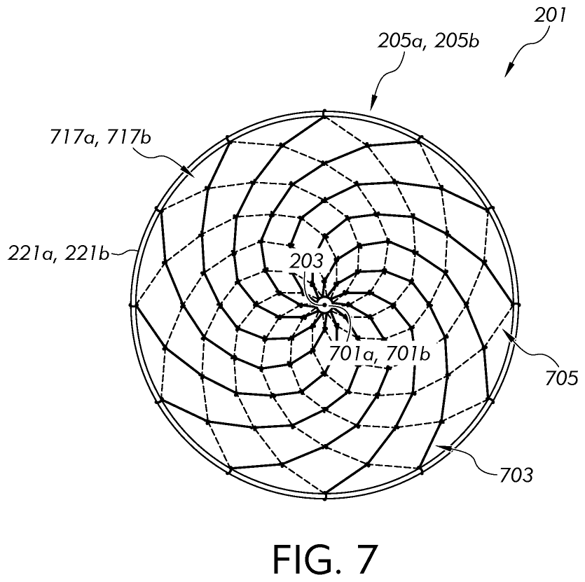

FIG. 6 illustrates features of a first lattice 617a and the second lattice 617b of sutures of the first and second axial ends 205a, 205b of the radially expandable frame 201 in accordance with another embodiment of the disclosure. As shown, the first lattice 617a of sutures can define a central first lattice opening 601a and the second lattice 617b of sutures can define a central second lattice opening 601b. Each central lattice opening 601a, 601b can be substantially concentric with the frame axis 203. A plurality of substantially straight central segments in tension can be aligned in a circular array to approximate a circular central lattice opening with the substantially straight segments. Although not shown, other central lattice opening shapes may be provided in further embodiments.

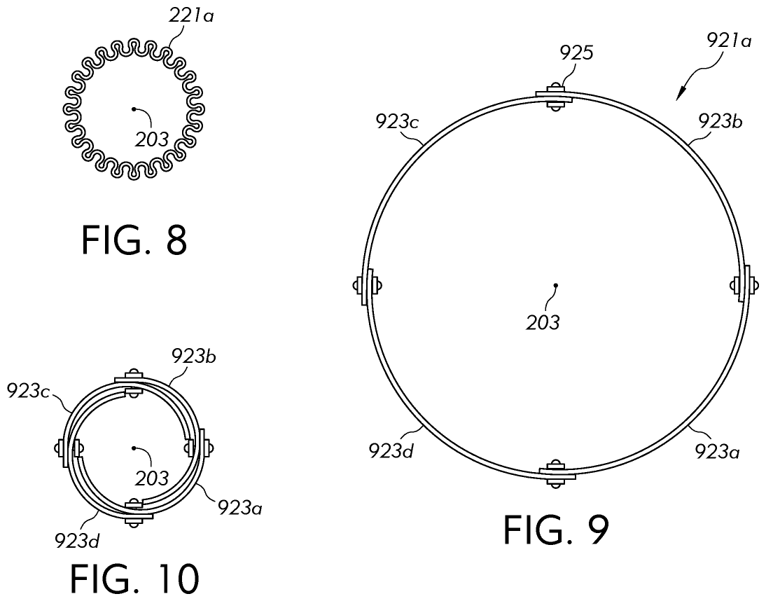

FIG. 7 illustrates features of a first lattice 717a and the second lattice 717b of sutures of the first and second axial ends 205a, 205b of the radially expandable frame 201 in accordance with another embodiment of the disclosure. As shown, the first lattice 717a of sutures can define a central first lattice opening 701a and the second lattice 717b of sutures can define a central second lattice opening 701b. Each central lattice opening 701a, 701b can be substantially concentric with the frame axis 203. Although not shown, other central lattice opening shapes may be provided in further embodiments.

Providing the first lattice 217a, 617a, 717a of sutures with the central first lattice opening 501a, 601a, 701a can be beneficial to provide an entrance opening for support material to be added by way of an injection tube passing through the central first lattice opening 501a, 601a, 701a. Furthermore, the compliant nature of the first lattice 217a, 617a, 717a of sutures can allow the diameter of the central first lattice opening 501a, 601a, 701a to quickly contract once the injection tube is removed after filling the interior area of the radially expandable frame with support material. As such, the central first lattice opening 501a, 601a, 701a can comprise diameter large enough to provide an injection tube that can fill the interior area of the radially expandable frame at a desired fill rate. Furthermore, the central first lattice opening 501a, 601a, 701a can automatically contract once the filling procedure is complete in response to removing the injection tube due to the tension and flexible nature of the sutures that are provided in the lattice arrangement. Filling of support material through the second axial end 205b may not require since the second axial end 205b will be facing the distal end of the left atrial appendage “LAA” as shown in FIG. 1. As such, in some embodiments, the second axial end 205b may not be provided with a second lattice 217b, 617b, 717b of sutures or the second lattice 217b, 617b, 717b of sutures may not comprise a central second lattice opening 501b, 601b, 701b. Rather, another feature may be provided at the second axial end that can prevent support material from passing through the second axial end without the need to accommodate an injection tube for filling the interior of the radially expandable frame.

With further reference to FIG. 5, the first lattice 217a of sutures and the second lattice 217b of sutures can further define a first array 505 of lattice openings 507 surrounding the frame axis 203. As shown, in some embodiments, the first array 505 can comprise a circular array of lattice openings 507 surrounding the frame axis 203. In some embodiments, the first array 505 of lattice openings 507 can comprise openings with identical shapes with identical or different sizes although different shapes may be provided in further embodiments. In addition or alternatively, the first array 505 of lattice openings 507 can comprise geometric shapes although other shapes may be provided in further embodiments. In some embodiments, the geometric shapes can comprise a polygonal shape with three or more sides. For example, as shown in FIG. 5, the first array 505 of lattice openings 507 each comprise the shape of a triangle with a base 503 of the arrayed triangles defining the corresponding central lattice opening.

With further reference to FIG. 5, the first lattice 217a of sutures and second lattice 217b of sutures can further define a second array 509 of lattice openings 511 surrounding the frame axis 203. As shown, in some embodiments, the second array 509 can comprise a circular array of lattice openings 511 surrounding the frame axis 203. In some embodiments, the second array 509 of lattice openings 511 can comprise openings with identical shapes with identical or different sizes although different shapes may be provided in further embodiments. In addition or alternatively, the second array 509 of lattice openings 511 can comprise geometric shapes although other shapes may be provided in further embodiments. In some embodiments, the geometric shapes can comprise a polygonal shape with three or more sides. For example, as shown in FIG. 5, the second array 509 of lattice openings 511 each comprise the shape of a parallelogram with approximately half of each parallelogram nested between a pair of adjacent triangular shaped openings 507 of the first array 505. Furthermore, one corner of the parallelogram touches the periphery of the central first and second lattice opening 501a, 501b while the opposite corner is attached to the first and second ring 221a, 221b.

As shown in FIG. 6, the first lattice 617a of sutures and the second lattice 617b of sutures can further define a first array 605 of lattice openings 607 surrounding the frame axis 203. As shown, in some embodiments, the first array 605 can comprise a circular array of lattice openings 607 surrounding the frame axis 203. In some embodiments, the first array 605 of lattice openings 607 can comprise openings with identical shapes with identical or different sizes although different shapes may be provided in further embodiments. In addition or alternatively, the first array 605 of lattice openings 607 can comprise geometric shapes although other shapes may be provided in further embodiments. In some embodiments, the geometric shapes can comprise a polygonal shape with three or more sides. For example, as shown in FIG. 6, the first array 605 of lattice openings 607 each comprise the shape of a triangle with a base 603 of the arrayed triangles defining the corresponding central lattice opening.

With further reference to FIG. 6, the first lattice 617a of sutures and second lattice 617b of sutures can further define a second array 609 of lattice openings 611 surrounding the frame axis 203. As shown, in some embodiments, the second array 609 can comprise a circular array of lattice openings 611 surrounding the frame axis 203. In some embodiments, the second array 609 of lattice openings 611 can comprise openings with identical shapes with identical or different sizes although different shapes may be provided in further embodiments. In addition or alternatively, the second array 609 of lattice openings 611 can comprise geometric shapes although other shapes may be provided in further embodiments. In some embodiments, the geometric shapes can comprise a polygonal shape with three or more sides. For example, as shown in FIG. 6, the second array 609 of lattice openings 611 each comprise the shape of a quadrilateral with a portion that is nested between a pair of adjacent triangular shaped openings 607 of the first array 605. Furthermore, one corner of the quadrilateral touches the periphery of the central first and second lattice opening 601a, 601b.

With further reference to FIG. 6, the first lattice 617a of sutures and the second lattice 617b of sutures can further define a third array 613 of lattice openings 615 surrounding the frame axis 203. As shown, in some embodiments, the third array 613 can comprise a circular array of lattice openings 615 surrounding the frame axis 203. In some embodiments, the third array 613 of lattice openings 615 can comprise openings with identical shapes with identical or different sizes although different shapes may be provided in further embodiments. In addition or alternatively, the third array 613 of lattice openings 615 can comprise geometric shapes although other shapes may be provided in further embodiments. In some embodiments, the geometric shapes can comprise a polygonal shape with three or more sides. For example, as shown in FIG. 6, the third array 613 of lattice openings 615 each comprise the shape of a triangle with the corners of the base of each triangle being attached to a corresponding corner of an adjacent triangle in the array and the apex of the triangle being attached to the first and second ring 221a, 221b. Furthermore, the apex of each triangle of the first array 605 can be attached to a midpoint of a base of a corresponding triangle of the third array 613. As shown in FIGS. 5 and 6, overlapping portions of sutures can be attached to one another. For example, overlapping portions of sutures can be attached to one another by being knotted together.

With further reference to FIG. 7, the first lattice 717a of sutures and the second lattice 717b of sutures can each be provided with a first set 703 of spiral sutures (shown in solid lines) that spiral in a clockwise direction about each central lattice opening 701a, 701b from the central lattice opening to the attachment location on the corresponding ring 221a, 221b. The number of clockwise spiral sutures can equal the number of attachment locations (e.g., apertures on the ring) with each clockwise spiral suture being attached to the central lattice opening and spirally wound about the opening in a clockwise fashion to be attached to a corresponding attachment location. Furthermore, the first lattice 717a of sutures and the second lattice 717b of sutures can each be provided with a second set 705 of spiral sutures (shown in broken lines) that spiral in a counterclockwise direction about each central lattice opening 701a, 701b from the central lattice opening to the attachment location on the corresponding ring 221a, 221b. The number of counterclockwise spiral sutures can equal the number of attachment locations (e.g., apertures on the ring) with each counterclockwise spiral suture being attached to the central lattice opening and spirally wound about the opening in a counterclockwise fashion to be attached to a corresponding attachment location. As shown, the spiral sutures can be attached to each other at points of overlap (e.g., by being knotted together).

FIGS. 1-3 and 5-10 illustrate features of the occlusion apparatus 101 without the support material for clarity. FIG. 4 illustrates a cross section of the occlusion apparatus 101 along line 4-4 of FIG. 2 and further illustrating support material 401 filling the interior area 209 of the radially expandable frame 201 to facilitate maintenance of the radially expandable frame 201 in the illustrated radially expanded orientation. As shown, the first lattice 217a of sutures inhibits (e.g., prevents) the support material 401 from axially exiting the interior area 209 through the first opening 219a. As further illustrated, the second lattice 217b of sutures inhibits (e.g., prevents) the support material 401 from axially exiting the interior area 209 through the second opening 219b. Still further, spaces between the longitudinal segments 207 and tabs 211 are small enough to inhibit (e.g., prevent) the support material 401 from radially exiting the interior area 209 through the sidewalls of the radially expandable frame 201. In some embodiments, the support material can comprise a gel. For instance, the gel can comprise a viscosity that is selected to be sufficiently high to prevent flowing of the gel through the lattices of sutures and/or openings in the sidewalls of the radially expandable frame. In some embodiments, the gel can comprise a storage modulus of greater than 150 Pa.

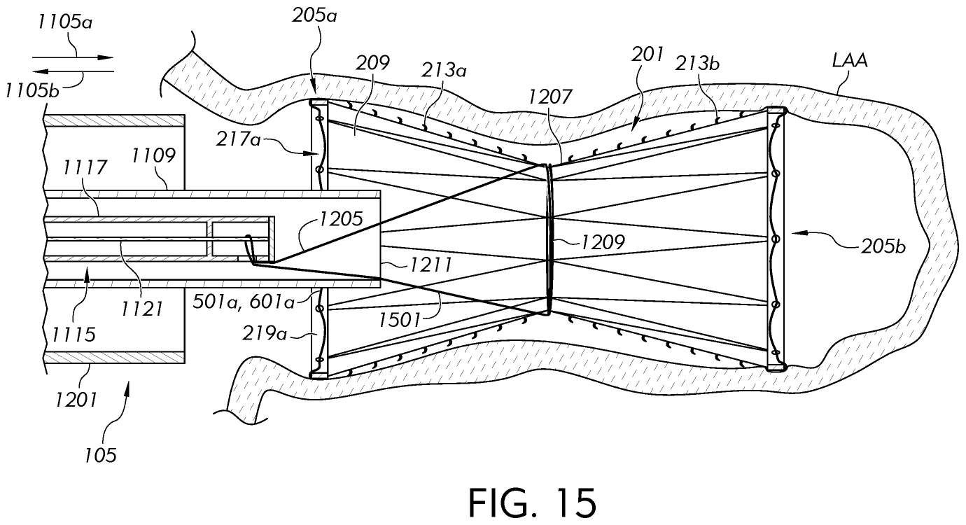

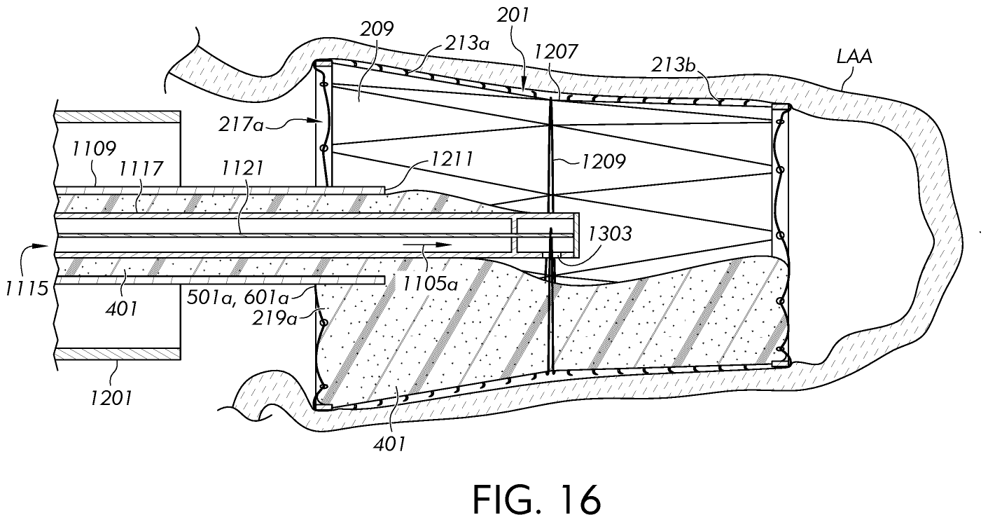

A delivery device 105 for deploying an occlusion apparatus 101 will be discussed schematically with initial reference to FIGS. 1, and 11-13. As shown in FIGS. 11-12, the delivery device comprises an outer tube 1101 attached at a distal end to the capsule 1201. A control grip 1103 on a handle apparatus 107 of the delivery device 105 may be provided at the proximal end of the outer tube 1101. The control grip 1103 can be moved by a surgeon in a distal direction 1105a to move the outer tube 1101 together with the capsule 1201 in the distal direction 1105a. Alternatively, the control grip 1103 can be moved by a surgeon in a proximal direction 1105b to move the outer tube 1101 together with the capsule 1201 in the proximal direction 1105b.

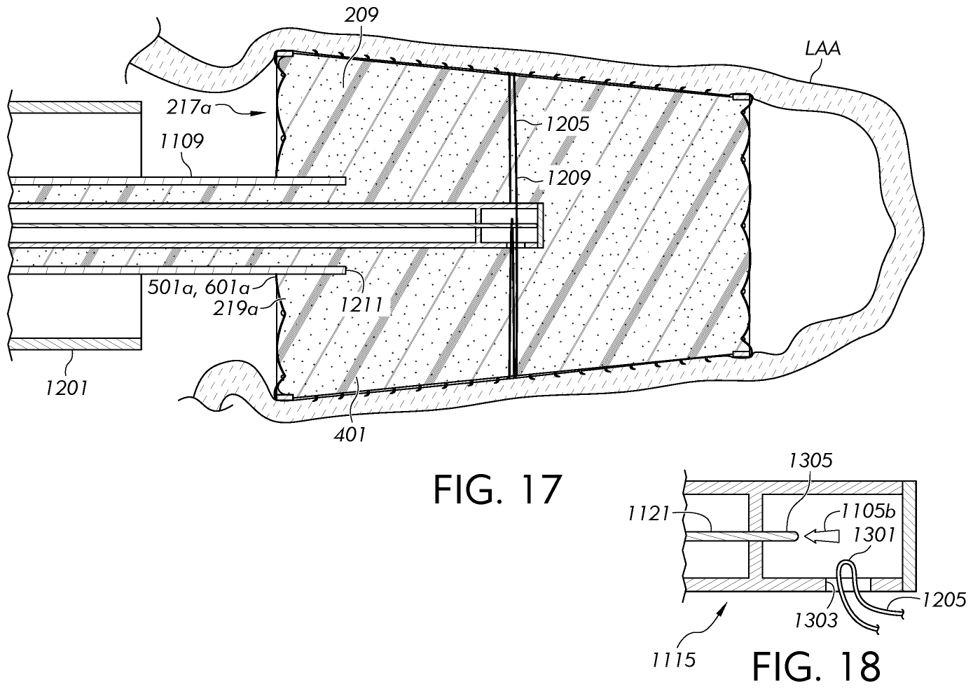

The delivery device 105 can further comprise an injection device 1107 including an injection tube 1109 extending within a lumen of the outer tube 1101. The injection device 1107 can include an inlet port 1111 connected to a source 1113 of support material. The delivery device 105 can further include a cinch device 1115 comprising an elongated cinch element 1117 received within a lumen of the injection tube 1109 and a release wire 1121 configured to releasably engage a portion of a cinch suture 1205 (see FIGS. 12-13). The handle apparatus 107 of the delivery device 105 can comprise a cinch grip 1119 configured to allow a surgeon to move the elongated cinch element 1117 together with the release wire 1121 relative to the injection tube 1109. The handle apparatus 107 of the delivery device 105 can further comprise a release grip 1123 configured to allow a surgeon to release the portion of the cinch suture 1205.

Methods of deploying the occlusion apparatus 101 with the delivery device 105 can begin with initially positioning the radially expandable frame 201 in a radially collapsed orientation within an interior area 1203 of the capsule 1201 of the delivery device 105 as shown in FIG. 12. As shown in FIG. 12, the cinch suture 1205 is loosely wound in one or more loops 1209 about a central waist 1207 of the collapsed radially expandable frame 201 wherein the central waist 1207 can comprise an outer diameter that is less than the outer diameters of the axial ends 205a, 205b. Furthermore, as shown in FIG. 13, a portion 1301 of the cinch suture 1205 can extend through an aperture 1303 of the elongated cinch element. As shown, the portion 1301 of the cinch suture 1205 is releasably engaged by a distal end portion 1305 of the release wire 1121. For instance, as shown, the distal end portion 1305 of the release wire 1121 can extend through the portion 1301 comprising a looped portion of the cinch suture 1205.

Methods of placing the occlusion apparatus 101 can comprise a transseptal approach wherein the introducer sheath 103 can be introduced into the right atrium “RA” from the inferior vena cava “IVC”. The introducer sheath 103 can then puncture through the coronary septum and into the left atrium “LA” and adjacent the left atrial appendage “LAA”. The capsule 1201 of the delivery device 105 containing the radially expandable frame 201 in the collapsed orientation (see FIG. 12) is then passed through the lumen of the introducer sheath 103 to be properly positioned within the left atrial appendage “LAA” as shown in FIG. 12. As schematically illustrated in FIG. 1, the delivery device 105 can comprise a handle apparatus 107 that can be manipulated by a surgeon outside of the patient's vasculature to control delivery and deployment of the occlusion apparatus 101.

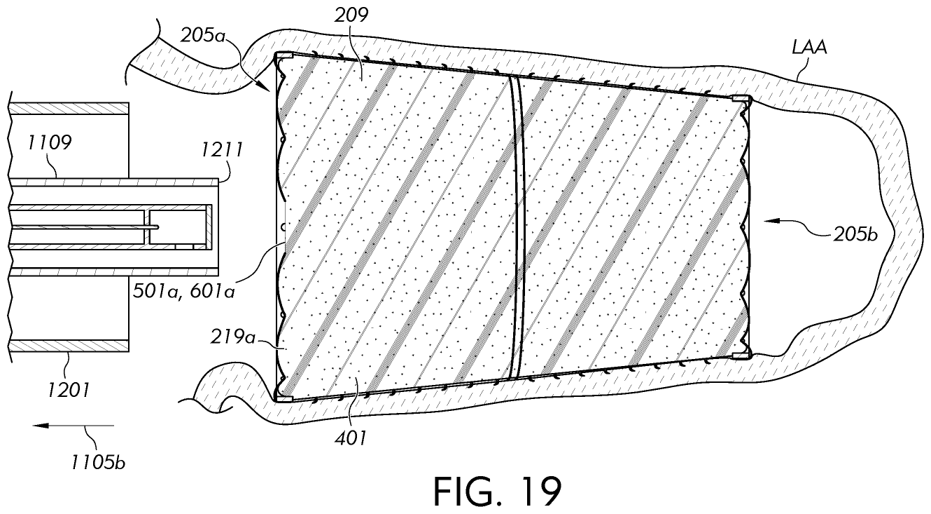

The position of the capsule 1201 can be monitored by X-rays using fluoroscopy to properly position the capsule at the entrance to the left atrial appendage “LAA”. Once positioned, as shown in FIG. 14, the surgeon may grasp the control grip 1103 of the handle apparatus 107 and pull the control grip 1103 in the proximal direction 1105b relative to the remaining components of the delivery device 105. As shown in FIG. 14, they capsule 1201 then shifts in the proximal direction 1105b relative to the radially expandable frame 201, the injection tube 1109, the elongated cinch element 1117, the cinch suture 1205, and the release wire 1121. The second axial end 205b then begins to self expand since it is no longer constrained from expanding within the interior of the capsule 1201. At the position shown in FIG. 14, the radially expandable frame 201 has not fully expanded and the protrusions 213a, 213b have not yet been anchored to the wall of the left atrial appendage “LAA”. As such, the position of the occlusion apparatus 101 can be further adjusted by the surgeon. In the position shown in FIG. 14, segments 1501 of the cinch suture 1205 may be loose and not under tension between the loops 1209 and the point of connection with the cinch device 1115 discussed with respect to FIG. 13.

As shown in FIG. 15, the capsule 1201 can be further retracted in the proximal direction 1105b relative to the radially expandable frame 201 such that the radially expandable frame 201 is positioned outside of the capsule 1201. The radially expandable frame 201 can then radially self expand from a radially collapsed orientation to a radially expanded orientation. For example, as shown in FIG. 15 the diameter of the first axial end 205a and the central waist 1207 of the radially expandable frame 201 self expands as the radially expandable frame 201 begins returning to the radially expanded orientation. As the diameter of the central waist 1207 increases, the loops 1209 of the cinch suture 1205 tighten and the slack in segments 1501 of the cinch suture 1205 is taken up and placed under tension to restrict further radial expansion of the radially expandable frame 201 to a first radially expanded position shown in FIG. 15. In the first radially expanded position shown in FIG. 15, a surgeon can still adjust the position of the occlusion apparatus 101 in the proximal and/or distal directions by adjusting the handle apparatus 107 since the protrusions 213a, 213b have not yet anchored into the wall of the left atrial appendage “LAA”.

After achieving the first radially expanded orientation illustrated in FIG. 15 and carrying out any final adjustments of the location of the occlusion apparatus 101 relative to the left atrial appendage “LAA”, the method can further comprise adjusting the cinch suture to further radially expand the radially expandable frame to a second radially expandable orientation. The cinch device 1115 can be designed to facilitate further radial expansion to the second radially expandable orientation. For example, as shown in FIG. 13, the distal end portion 1305 of the release wire 1121 can extend through the portion 1301 comprising a looped portion of the cinch suture 1205. The release wire 1121 can therefore secure the cinch suture 1205 to the distal end portion of the elongated cinch element 1117 of the cinch device 1115. The cinch device 1115 can be used to adjust the cinch suture 1205 to further radially expand the radially expandable frame 201 to the second radially expandable orientation. Adjusting the cinch suture can comprise distally extending the cinch device to reduce a distance between the secured location and a location of the radially expandable frame where the cinch suture restricts the radial expansion of the radially expandable frame. For instance, the surgeon can grasp the cinch grip 1119 and push the cinch grip in the distal direction 1105a to cause the elongated cinch element 1117 and the release wire 1121 to likewise move together in the distal direction 1105a relative to the injection tube 1109 and the capsule 1201. As the portion 1301 of the cinch suture 1205 that is attached at the secured location to the distal end portion of the elongated cinch element 1117 moves towards the loops 1209, the segments 1501 are taken up by the loops 1209 to allow the loops 1209 to dilate. The dilating loops 1209 allow the central waist 1207 of the radially expandable frame 201 to self-expand until the self-expansion is again limited by the tension within the dilated loops 1209 as shown in FIG. 16. In the position shown in FIG. 16, the protrusions 213a, 213b can begin to penetrate into the wall of the left atrial appendage “LAA” to secure the selected position of the occlusion apparatus 101 relative to the left atrial appendage “LAA.”

As shown in FIG. 16, the method can further include filling an interior area 209 of the radially expandable frame 201 with a support material 401 to facilitate maintenance of the radially expandable frame 201 in the radially expanded orientation. As shown in FIGS. 12 and 14-17, the delivery device 105 can further comprise an injection device 1107 comprising an injection tube 1109 extending through the first opening 219a of the radially expandable frame 201. As shown, a distal end 1211 of the injection tube 1109 positioned within the interior area 209 of the radially expandable frame 201. As shown in FIG. 12, in some embodiments, a radial circumferential travel path 1215 can be defined between an inner circumferential surface of the injection tube 1109 and an outer circumferential surface of the elongated cinch element 1117. The radial circumferential travel path 1215 can be in communication with a passage 1125 of the inlet port 1111. The injection tube 1109 and the elongated cinch element 1117.

In some embodiments, the distal end 1211 of the injection tube 1109 can be positioned within the interior area 209 of the radially expandable frame 201 while the radially expandable frame 201 is collapsed and positioned within the capsule 1201 and delivered to a patient's vasculature to the left atrial appendage “LAA” as shown in FIG. 12. The distal end 1211 of injection tube 1109 can also remain within the interior area 209 of the radially expandable frame 201 while retracting the capsule 1201 in the proximal direction 1105b, while radially expanding the radially expandable frame 201, and while filling the radially expandable frame 201 with support material 401 as shown in FIGS. 14-17. As further illustrated, in some embodiments, the distal end 1211 of the injection tube 1109 can remain within the interior area 209 of the radially expandable frame 201 while also remaining stationary relative to the radially expandable frame 201 while retracting the capsule 1201 in the proximal direction 1105b, while radially expanding the radially expandable frame 201, and while filling the radially expandable frame 201 with support material 401 as shown in FIGS. 14-17. Arranging the distal end to remain stationary relative to the radially expandable frame, as shown in FIGS. 14-17, can help prevent inadvertent removal of the distal end 1211 within the interior area 209 before the interior area 209 is filled with support material 401.

As further illustrated in FIGS. 12 and 14-17, the first lattice 217a, 617a of sutures extends across the first opening 219a while the injection tube 1109 extends through the first opening 219a of the radially expandable frame 201. In some embodiments, the lattice of sutures can provide a benefit in that the injection tube 1109 may extend through one or more openings in the lattice to permit the distal end 1211 of the injection tube 1109 to extend within the interior area 209 while the first lattice 217a, 617a of sutures extends across the first opening 219a. the injection tube further extends through a central first lattice opening 501a, 601a of the first lattice 217a, 617a of sutures when passing support material 401 through the injection tube 1109.

As shown in FIGS. 16-17, the interior area 209 of the radially expandable frame 201 can be partially or entirely filled with support material 401. In some embodiments, the support material can comprise gel. In some embodiments, the gel can cross-link to become a solid interior plug. Providing a gel that cross-links can help retain the gel within the interior area while only a minimal level of suture coverage across the opening(s) into the radially expandable frame. In further embodiments, the gel can comprise a viscous gel that maintains a significant viscosity (or retains substantially the same viscosity) over time. Providing a viscous gel can allow the occlusion apparatus to change shape over time to match a changed shape of the left atrial appendage “LAA.” In some embodiments, the gel can comprise a storage modulus of greater than or equal to 150 Pa although other materials may be incorporated with a storage modulus less than 150 Pa in further embodiments.

Filling the interior area 209 of the radially expandable frame 201 comprises passing the support material 401 through the injection tube 1109. For example, a control valve (not shown) can be activated to cause support material 401 to be delivered from the source 1113 of support material 401 (see FIG. 11). The support material 401 can pass through a conduit 1127 and into the passage 1125 of the inlet port 1111 from an end portion 1129 of the conduit 1127. The support material 401 can then travel through the radial circumferential travel path 1215 and then pass through the distal end 1211 of the injection tube 1109 and into the interior area 209 of the radially expandable frame 201 as shown in FIG. 16.

In some embodiments, filling of the interior area 209 can occur after partially or entirely radially expanding the radially expandable frame 201. In further embodiments, as shown, filling the interior area 209 can occur after partially radially expanding and can further occur while continuing to radially expand to the final radially expanded orientation. For example, filling the interior area of the radially expandable frame with the support material occurs after radially expanding the radially expandable frame. In the illustrated embodiment, filling the interior area 209 with the support material 401 can occur while adjusting the diameter of the loops 1209 from the diameter shown in FIG. 15 to the diameter shown in FIG. 16 or after adjusting the diameter of the loops 1209. Although not shown, in some embodiments, the fillable material can begin filling the interior area while the radially expandable frame 201 is still in the collapsed orientation within the capsule 1201 or anytime during or after retracting the capsule 1201 in the proximal direction.

In some embodiments, as shown in FIG. 17, filling of the interior area 209 with support material 401 can continue until the support material 401 becomes pressurized and forces the radially expandable frame 201 to further expand against the tension of the loops 1209 of the cinch suture 1205 to the final radially expanded orientation shown in FIG. 17. As shown, radial expansion can cause the protrusions 213a, 213b to be embedded within the wall of the left atrial appendage “LAA”, thereby anchoring the occlusion apparatus 101 in the desired position. After filling the interior area 209 of the radially expandable frame 201 with the support material 401, the tension within the cinch suture 1205 can be released. For example, once the surgeon is ready to release the cinch suture 1205, the surgeon can grasp the release grip 1123 of the handle apparatus 107 (see FIG. 11) and pull the release grip 1123 in the proximal direction 1105b. Pulling the release grip 1123 in the proximal direction can retract the release wire 1121 in the proximal direction 1105b to cause the distal end portion 1305 of the release wire 1121 to release the loop portion 1301 of the cinch suture 1205, and thereby release the tension in the cinch suture 1205. The loop portion 1301 can then pass through the aperture 1303 wherein the cinch suture 1205 can remain with the implanted occlusion apparatus 101. The cinch suture 1205 may be designed to dissolve over time.

After filling the interior area 209 of the radially expandable frame 201 with the support material 401, as shown in FIG. 19, the injection tube 1109 and the remaining components of the delivery device 105 can be retracted in the proximal direction 1105b until the distal end 1211 of the injection tube 1109 is positioned outside of the interior area 209 of the radially expandable frame 201. Furthermore, in some embodiments, after positioning the distal end 1211 of the injection tube 1109 outside of the interior area 209 of the radially expandable frame 201, the central first lattice opening 501a, 601a can contract to a reduced size to inhibit support material 401 from exiting the interior area 209 through the central first lattice opening 501a, 601a. The flexible lattice therefore provides the benefit of allowing a self adjusting sized opening to accommodate a relatively large diameter injection tube 1109 for quickly filling the interior area 209 with support material 401 while quickly automatically reducing in size after removing the injection tube 1109 that helps provide an ideal sized opening to prevent passage of the support material 401 through the retracted central first lattice opening 501a, 601a. Furthermore, during and after filling the interior area 209 with support material 401, the first lattice 217a of sutures can retain support material 401 within the interior area 209 and prevent support material 401 from exiting the interior area 209 through the first opening 219a. Likewise, during and after filling the interior area 209 with support material 401, the first lattice 217a of sutures can retain support material 401 within the interior area 209 and prevent support material 401 from exiting the interior area 209 through the second opening 219b.

As shown in FIG. 19, once the radially expandable frame 201 is completely filled with support material 401, the occlusion apparatus 301 can assume a frustoconical shape with the first axial end 205a having a greater diameter than the second axial end 205b to help prevent inserting the occlusion apparatus 301 too far into the left atrial appendage “LAA” where an additional undesired cavity may form between the left atrium “LA” and the first axial end 205a that may result in to thrombi formation that the occlusion apparatus was designed to prevent. In some embodiments, each protrusion 213b of the second set of the plurality of protrusions with the distal end extending in a direction towards the second axial end 205b can further help maintain the installed position of the radially expandable frame 201 by also resisting movement of the radially expandable frame 201 further into the left atrial appendage “LAA”. In some embodiments, each protrusion 213a of the first set of the plurality of protrusions provides the distal end extending in the direction towards the first axial end 205a to help maintain the installed position of the radially expandable frame 201 by resisting movement of the radially expandable frame 201 out of the left atrial appendage “LAA” and into the left atrium “LA”.

In accordance with the disclosure, non-limiting aspects of the disclosure will now be described. Various combinations of the aspects can be provided in accordance with the disclosure.

Aspect 1. An occlusion apparatus comprises a radially expandable frame extending along a frame axis from a first axial end to a second axial end. The occlusion apparatus further comprises a first lattice of sutures extending across a first opening defined by the first axial end of the radially expandable frame.

Aspect 2. The occlusion apparatus of aspect 1, further comprising support material filling an interior area of the radially expandable frame to facilitate maintenance of the radially expandable frame in a radially expanded orientation. The first lattice of sutures inhibits the support material from exiting the interior area through the first opening.

Aspect 3. The occlusion apparatus of aspect 2, wherein the support material comprises gel.

Aspect 4. The occlusion apparatus of aspect 3, wherein the gel comprises a storage modulus of greater than 150 Pa.

Aspect 5. The occlusion apparatus of any one of aspects 1-4, wherein the first lattice of sutures defines a central first lattice opening that is substantially concentric with the frame axis.

Aspect 6. The occlusion apparatus of any one of aspects 1-5, wherein the first lattice of sutures defines a first array of lattice openings surrounding the frame axis.

Aspect 7. The occlusion apparatus of aspect 6, wherein the first array of openings comprise openings with identical shapes.

Aspect 8. The occlusion apparatus of any one of aspects 6-7, wherein the first array of openings comprise openings that each comprise a geometric shape.

Aspect 9. The occlusion apparatus of aspect 8, wherein the geometric shape comprises the shape of a triangle.

Aspect 10. The occlusion apparatus of aspect 8, wherein the geometric shape comprises the shape of a parallelogram.

Aspect 11. The occlusion apparatus of any one of aspects 2-10, further comprising a second lattice of sutures extending across a second opening defined by the second axial end of the radially expandable frame. The second lattice of sutures inhibits the support material from exiting the interior area through the second opening.

Aspect 12. The occlusion apparatus of aspect 11, wherein the second lattice of sutures defines a central second lattice opening that is substantially concentric with the frame axis.

Aspect 13. The occlusion apparatus of any one of aspects 11-12, wherein the second lattice of sutures defines a first array of lattice openings surrounding the frame axis.

Aspect 14. The occlusion apparatus of aspect 13, wherein the second array of openings comprise openings with identical shapes.

Aspect 15. The occlusion apparatus of any one of aspects 13-14, wherein the second array of openings comprise openings that each comprise a geometric shape.

Aspect 16. The occlusion apparatus of aspect 15, wherein the geometric shape of each opening of the second array of openings comprises the shape of a triangle.

Aspect 17. The occlusion apparatus of aspect 15, wherein the geometric shape of each opening of the second array of openings comprises the shape parallelogram.

Aspect 18. The occlusion apparatus of any one of aspects 1-10, wherein the first axial end comprises a first ring defining the first opening, wherein the first lattice of sutures are attached to the first ring at a plurality of first ring locations that are circumferentially arranged about the frame axis.

Aspect 19. The occlusion apparatus of any one of aspects 11-17, wherein the first axial end comprises a first ring defining the first opening, wherein the first lattice of sutures are attached to the first ring at a plurality of first ring locations that are circumferentially arranged about the frame axis.

Aspect 20. The occlusion apparatus of aspect 19, wherein the second axial end comprises a second ring defining the second opening, wherein the second lattice of sutures are attached to the second ring at a plurality of second ring locations that are circumferentially arranged about the frame axis.

Aspect 21. The occlusion apparatus of aspect 20, wherein the second ring has a ring stiffness that is greater than a ring stiffness of the first ring.

Aspect 22. The occlusion apparatus of any one of aspects 1-20, wherein the first ring comprises a plurality of linked segments.

Aspect 23. The occlusion apparatus of any one of aspects 1-17, wherein the first axial end comprises a plurality of crowns circumferentially arranged about the frame axis, wherein the first lattice of sutures are threaded through the plurality of crowns.

Aspect 24. The occlusion apparatus of any one of aspects 1-23, wherein the radially expandable frame comprises a frustoconical shape.

Aspect 25. The occlusion apparatus of aspect 24, wherein the frustoconical shape tapers in a direction from the first axial end towards the second axial end.

Aspect 26. The occlusion apparatus of any one of aspects 1-25, wherein the radially expandable frame comprises a plurality of protrusions extending away from the frame axis and an exterior of the radially expandable frame.

Aspect 27. The occlusion apparatus of aspect 26, wherein the plurality of protrusions each comprise a hook.

Aspect 28. The occlusion apparatus of any one of aspects 26-27, wherein each protrusion of a first set of the plurality of protrusions comprises a distal end extending in a direction towards the first axial end.

Aspect 29. The occlusion apparatus of any one of aspects 26-28, wherein each protrusion of a second set of the plurality of protrusions comprises a distal end extending in a direction towards the second axial end.

Aspect 30. A method of deploying an occlusion apparatus comprising a radially expandable frame extending along a frame axis from a first axial end to a second axial end. The occlusion apparatus further comprises a first lattice of sutures extending across a first opening defined by the first axial end of the radially expandable frame. The radially expandable frame is positioned in a radially collapsed orientation within an interior area of a capsule of a delivery device. The method comprises retracting the capsule in a proximal direction relative to the radially expandable frame such that the radially expandable frame is positioned outside of the capsule. The method further comprises radially expanding the radially expandable frame from the radially collapsed orientation to a radially expanded orientation. The method further comprises filling an interior area of the radially expandable frame with a support material to facilitate maintenance of the radially expandable frame in the radially expanded orientation. The first lattice of sutures inhibits the support material from exiting the interior area through the first opening.

Aspect 31. The method of aspect 30, wherein the delivery device further comprises an injection device comprising an injection tube extending through the first opening of the radially expandable frame with a distal end of the injection tube positioned within the interior area of the radially expandable frame, wherein filling the interior area of the radially expandable frame comprises passing the support material through the injection tube.

Aspect 32. The method of aspect 31, wherein the distal end of the injection tube is positioned within the interior area of the radially expandable frame while retracting the capsule in the proximal direction.

Aspect 33. The method of any one of aspects 31-32, wherein the injection tube further extends through a central first lattice opening of the first lattice of sutures when passing support material through the injection tube.

Aspect 34. The method of aspect 33, wherein, after filling the interior area of the radially expandable frame with the support material, the injection tube is retracted until the distal end of the injection tube is positioned outside of the interior area of the radially expandable frame. Furthermore, after positioning the distal end of the injection tube outside of the interior area of the distal frame, the central first lattice opening contracts to a reduced size to inhibit support material from exiting the interior area through the central first lattice opening.

Aspect 35. The method of any one of aspects 30-34, wherein filling the interior area of the radially expandable frame with the support material occurs after radially expanding the radially expandable frame.

Aspect 36. The method of any one of claims 30-35, wherein radially expanding the radially expandable frame occurs by self-expanding.

Aspect 37. The method of any one of aspects 30-36, wherein after retracting the capsule in the proximal direction such that the radially expandable frame is positioned outside of the capsule, a cinch suture restricts the radial expansion of the radially expandable frame to a first radially expanded orientation.

Aspect 38. The method of aspect 37, wherein, after achieving the first radially expanded orientation, further comprising adjusting the cinch suture to further radially expand the radially expandable frame to a second radially expandable orientation.

Aspect 39. The method of aspect 38, wherein a cinch device is releasably secured to a portion of the cinch suture at a secured location and adjusting the cinch suture comprises distally extending the cinch device to reduce a distance between the secured location and a location of the radially expandable frame where the cinch suture restricts the radial expansion of the radially expandable frame.

Aspect 40. The method of any one of aspects 37-39, wherein filling the interior area of the radially expandable frame with a support material further radially expands the radially expandable frame to a final radially expandable orientation.

Aspect 41. The method of aspect 40, wherein after filling the interior area of the radially expandable frame with the support material, further comprising releasing a tension in the cinch suture.

Aspect 42. The method of aspect 41, further comprising proximally retracting a release wire to release the tension in the cinch suture.

Aspect 43. The method of any one of aspects 30-42, wherein the support material comprises gel.

Aspect 44. The method of aspect 43, wherein the gel comprises a storage modulus of greater than 150 Pa.

It should be understood that while various aspects have been described in detail relative to certain illustrative and specific examples thereof, the present disclosure should not be considered limited to such, as numerous modifications and combinations of the disclosed features are possible without departing from the scope of the following claims.

A delivery path of the radially expandable occlusion apparatus can comprise various routes well known in the art. Preferably, the path comprises the radially expandable occlusion apparatus being percutaneously passed through a body lumen to access the heart. Preferably, the body lumen comprises the right femoral vein.

What is claimed is:

1. An occlusion apparatus comprising:
a radially expandable frame extending along a frame axis from a first axial end to a second axial end; and
a first lattice of sutures extending across a first opening defined by the first axial end of the radially expandable frame.

2. The occlusion apparatus of claim 1, further comprising support material filling an interior area of the radially expandable frame to facilitate maintenance of the radially expandable frame in a radially expanded orientation, wherein the first lattice of sutures inhibits the support material from exiting the interior area through the first opening.

3. The occlusion apparatus of claim 2, further comprising a second lattice of sutures extending across a second opening defined by the second axial end of the radially expandable frame, wherein the second lattice of sutures inhibits the support material from exiting the interior area through the second opening.

4. The occlusion apparatus of claim 2, wherein the support material comprises gel.

5. The occlusion apparatus of claim 1, wherein the first lattice of sutures defines a central first lattice opening that is substantially concentric with the frame axis.

6. The occlusion apparatus of claim 1, wherein the radially expandable frame comprises a frustoconical shape.

7. The occlusion apparatus of claim 1, wherein the radially expandable frame comprises a plurality of protrusions extending away from the frame axis and an exterior of the radially expandable frame.

8. The occlusion apparatus of claim 7, wherein the plurality of protrusions each comprise a hook.

9. The occlusion apparatus of claim 8, wherein each protrusion of a first set of the plurality of protrusions comprises a distal end extending in a direction towards the first axial end.

10. A method of deploying an occlusion apparatus comprising a radially expandable frame extending along a frame axis from a first axial end to a second axial end, and a first lattice of sutures extending across a first opening defined by the first axial end of the radially expandable frame, the radially expandable frame positioned in a radially collapsed orientation within an interior area of a capsule of a delivery device, the method comprising:
retracting the capsule in a proximal direction relative to the radially expandable frame such that the radially expandable frame is positioned outside of the capsule;
radially expanding the radially expandable frame from the radially collapsed orientation to a radially expanded orientation; and
filling an interior area of the radially expandable frame with a support material to facilitate maintenance of the radially expandable frame in the radially expanded orientation, wherein the first lattice of sutures inhibits the support material from exiting the interior area through the first opening.

11. The method of claim 10, wherein the delivery device further comprises an injection device comprising an injection tube extending through the first opening of the radially expandable frame with a distal end of the injection tube positioned within the interior area of the radially expandable frame, wherein filling the interior area of the radially expandable frame comprises passing the support material through the injection tube.

12. The method of claim 11, wherein the injection tube further extends through a central first lattice opening of the first lattice of sutures when passing support material through the injection tube.

13. The method of claim 12, wherein, after filling the interior area of the radially expandable frame with the support material, the injection tube is retracted until the distal end of the injection tube is positioned outside of the interior area of the radially expandable frame, and after positioning the distal end of the injection tube outside of the interior area of the distal frame, the central first lattice opening contracts to a reduced size to inhibit support material from exiting the interior area through the central first lattice opening.

14. The method of claim 10, wherein filling the interior area of the radially expandable frame with the support material occurs after radially expanding the radially expandable frame.

15. The method of claim 10, wherein after retracting the capsule in the proximal direction such that the radially expandable frame is positioned outside of the capsule, a cinch suture restricts the radial expansion of the radially expandable frame to a first radially expanded orientation.

16. The method of claim 15, wherein, after achieving the first radially expanded orientation, further comprising adjusting the cinch suture to further radially expand the radially expandable frame to a second radially expandable orientation.

17. The method of claim 16, wherein a cinch device is releasably secured to a portion of the cinch suture at a secured location and adjusting the cinch suture comprises distally extending the cinch device to reduce a distance between the secured location and a location of the radially expandable frame where the cinch suture restricts the radial expansion of the radially expandable frame.

18. The method of claim 15, wherein filling the interior area of the radially expandable frame with a support material further radially expands the radially expandable frame to a final radially expandable orientation.

19. The method of claim 18, wherein after filling the interior area of the radially expandable frame with the support material, further comprising releasing a tension in the cinch suture.

20. The method of claim 10, wherein the support material comprises gel.
